(19) United States
(12) Patent Application Publication
(10) Pub. No.: US 2012/0184993 A1
(43) Pub. Date: Jul. 19, 2012

(54) EXPANDABLE FACET SCREW

(75) Inventors: Jared Arambula, San Diego, CA (US); Andrew Todd Mahar, San Diego, CA (US)

(73) Assignee: Alphatec Spine, Inc., Carlsbad, CA (US)

(21) Appl. No.: 13/350,524

(22) Filed: Jan. 13, 2012

Related U.S. Application Data
(60) Provisional application No. 61/432,658, filed on Jan. 14, 2011.

(51) Int. Cl. A61B 17/70; A61B 17/88

(52) U.S. Cl. 606/246; 606/279

(57) ABSTRACT

An exemplary facet screw for joining a first facet of a first vertebra with a second facet of a second vertebra includes proximal and distal ends. The proximal end includes a threaded region for attachment to the first facet. The distal end includes a non-threaded region with a first configuration that freely inserts into a bore of the second facet and a second configuration that partially interferes with the bore to retain the distal end within the second facet.

### CROSS-REFERENCE TO RELATED APPLICATIONS

This application claims priority to U.S. Provisional Application 61/432,658, filed Jan. 14, 2011, which is incorporated by reference herein in its entirety.

### FIELD

The present invention relates generally to a device for securely attaching bones together and, more specifically relates to a facet screw having features that facilitate improved relative attachment of vertebra through the facets.

### BACKGROUND

Different types of bones can have significant variation in terms of density and/or hardness. For example, a bone may be considered to be a dense and/or hard bone, commonly referred to as a cortical bone. Alternatively, a bone may be less dense and/or spongy, commonly referred to as a cancellous bone. Some bones can have regions that are relatively cortical and other regions that are relatively cancellous. For example, referring to FIG. 1A, a human thigh bone, or femur 40, has a central region 42 that is relatively cortical and end regions 44 that are relatively cancellous.

Due to damage of a bone or connecting tissue around a bone, both human and animal bones may require secured attachment relative to one another. Use of a bone screw is a known method for securely attaching a human or animal bone to another human or animal bone. Alternatively, a fractured bone, for example, a hip fracture, a broken leg bone, or a broken arm bone may require secured attachment to itself. Use of a bone screw is also known for securely attaching the fractured bone to itself. Referring to FIG. 1A, for example, the femur 40 is illustrated having a fracture 46 near the end region 44. In one known method of securement, for example, a bone screw 48 can be used to securely attach the femur 40 to itself across the fracture 46.

Other types of bones may also require secured attachment to one another or across fractures therein. In humans the vertebral column (commonly known as a backbone or spine) may often require such securement. The human vertebral column includes 33 individual bones or vertebrae that are customarily described in terms of being in one of five vertically distinct regions. The vertebrae in the lowest two regions of the vertebral column are fused together and therefore do not move relative to each other, i.e., the lowest two regions of the vertebral column are non-articulating. The vertebrae in the upper three regions of the vertebral column, however, are articulable and are adjacently joined via joints as will be described further hereinbelow.

The upper vertebrae (including the five Lumbar L1-L5 vertebrae, the twelve Thoracic T1-T12 vertebrae, and the seven Cervical C1-C7 vertebrae) have articulable joints that can be attached to adjacent vertebrae. The joints provide the backbone with flexibility and cushion against impact. Due to damage of a vertebra or tissue within or surrounding a vertebral joint, secured attachment or “fixation” of the vertebral joint may become necessary. A common method for securely attaching vertebrae across a joint is the insertion of a screw or screws threaded through the vertebral bone across the joint.

Culbert U.S. Patent Application Publication 2005/0216026 summarizes several vertebrae securement or fixation methods that use screws. Referring to FIGS. 1B and 1C from Culbert, the human vertebra includes a vertebral body 50 and a branched bony structure 52 attached to the vertebral body 50 by a pair of regions known as pedicles 54. The branched bony structure 52, the pedicles 54, and the vertebral body 50 define a spinal aperture 56 for accommodation of a human spinal cord (not shown). The branched bony structure 52 includes several regions to which medical practitioners have assigned names, for example, a pair of transverse processes 58, a spinous process 60, a pair of superior facets 62, and a pair of inferior facets 64.

Referring to FIG. 1B, an upper illustrated vertebra 66A includes the pair of superior facets 62A and the pair of inferior facets 64A. A lower illustrated vertebra 66B includes the pair of superior facets 62B and the pair of inferior facets 64B. The superior facets 62B of the lower illustrated vertebra 66B form facet joints 68 (See FIG. 1C) with the inferior facets 64A of the upper illustrated vertebra 66A.

In one method for securing a first vertebra, for example, the vertebra 66A to a second vertebra, for example, the vertebra 66B, a screw 70 is threaded through the facets 62B and 64A on each lateral side of the vertebra 66B and 66A, respectively, thereby securely attaching the facet joints 68. Such facet fixation for securing adjacent vertebrae was first described in 1948 by King. A modification to King's technique was described by Boucher in 1959. The Boucher technique directs the screw 70 through the inferior facet 64 of an upper vertebra 66A, through the superior facet 62 of an adjacent lower vertebra 66B and into the pedicle 54 on the adjacent lower vertebra 66B, as illustrated in FIGS. 1B and 1C.

Although the Boucher technique is illustrated as an example of bone to bone fixation using a screw, other techniques are known in the art for attachments to vertebrae as well as other bones, both human and non-human. In human patients suffering from bone mass loss, for example, osteoporotic and osteopenic patients, the loss of bone density often causes weakened and brittle bones. A bone screw may be less effective in maintaining a grasp on bone of decreased density as compared to bone having normal density. Fixation of bones having decreased density can therefore be problematic, resulting in slippage of the screw within the bone and ultimate loosening of the fixation. Therefore, a need exists for a bone screw that can provide and maintain an improved grasp on any type of bone, whether dense or spongy, but particularly in osteoporotic and osteopenic human patients. A bone screw as described herein benefits from being applicable to the fixation of any sort of human or non-human bone to any other sort of human or non-human bone.

### SUMMARY

An exemplary facet screw for joining a first facet of a first vertebra with a second facet of a second vertebra includes proximal and distal ends. The proximal end includes a threaded region for attachment to the first facet. The distal end includes a non-threaded region with a first configuration that freely inserts into a bore of the second facet and a second configuration that partially interferes with the bore to retain the distal end within the second facet.

In other features, a cannula extends from the proximal end to the distal end. A deployment member extends through the cannula and applies a compressive force to the distal end to transition the non-threaded region from the first configuration to the second configuration. The deployment member removably attaches to the distal end. The deployment member includes an external thread that engages with an internal thread of the cannula. The deployment member includes a radial protrusion that engages with a groove of the cannula.

In still other features, the threaded region and the non-threaded region comprise different materials. In yet other features, the threaded region and the non-threaded region comprise a common material. The non-threaded region is detachable from the threaded region.

In still other features, the facet screw includes a head on the proximal end for driving the facet screw into the first and second facets. A washer is coupled to the proximal end and includes a protrusion extending distally for engagement with the first facet. The non-threaded region includes an expandable region and an attachment region distal to the expandable region. The expandable region includes a plurality of struts forming an expandable cage. The expandable cage directs flow of bone material injected into the second bore in the second configuration.

An exemplary facet screw having a proximal end and a distal end includes a cannulated shaft, a washer, and a deployment member. The cannulated shaft includes a head, an externally threaded region, an expandable region, and an attachment region. The head is disposed on the proximal end and includes a diameter larger than a diameter of the cannulated shaft. The externally threaded region is distal to the head and configured for attachment to an inferior facet of a superior vertebra. The expandable region is distal to the externally threaded region and configured for insertion into bore of an adjacent superior facet of an inferior vertebra. The attachment region is distal to the expandable region. The washer is coupled to the cannulated shaft distal to the head and includes a protrusion extending from a distal end for engagement with the inferior facet. The deployment member includes a proximal portion and a distal portion. The proximal portion extends proximally from the head. The distal portion extends through the cannulated shaft and attaches to the attachment region. The deployment member applies a proximally directed force to compress the cannulated shaft and expand a portion of the expandable region.

An exemplary method of joining a first facet of a first vertebra with a second facet of a second vertebra includes the steps of aligning the first facet with the second facet, forming a first bore in the first facet, and forming a second bore in the second facet. The method further includes the steps of forming a thread pattern on the first bore and inserting a facet screw having a threaded region that engages the thread pattern of the first bore and a non-threaded region that freely inserts into the second bore. The method further includes the step of expanding the non-threaded region to partially interfere with the second bore and retain the distal end within the second facet.

In other features, the method further includes the step of inserting a deployment member through a cannula in the facet screw and applying a compressive force to expand the non-threaded region. The step of expanding the non-threaded region includes increasing a diameter of the non-threaded region from a first diameter that is less than a diameter of the second bore to a second diameter that is greater than or equal to the diameter of the second bore. The method further includes the step of injecting bone material through a cannula in the facet screw into the second bore. The method further includes the step of injecting bone material between the first facet and the second facet.

### BRIEF DESCRIPTION OF THE FIGURES

FIG. 1A is side elevation of a femur attached to itself across a fracture using a screw.

FIG. 1B is a rear view of two human vertebra fixed together via the Boucher Technique.

FIG. 1C is a view looking up at the bones of FIG. 1B from below.

FIG. 2 is an isometric view of an embodiment of a facet screw of the present invention.

FIG. 3 illustrates a side view of an embodiment of a cannulated shaft.

FIG. 4A illustrates a side view of an embodiment of a washer.

FIG. 4B illustrates a plan view of the washer of FIG. 4A.

FIG. 5 illustrates a cross-sectional view of the cannulated shaft of FIG. 3, taken generally along the line 5-5 of FIG. 3.

FIG. 6 illustrates a side view of another embodiment of a cannulated shaft.

FIG. 7 illustrates a side view of a further embodiment of a cannulated shaft.

FIG. 8A illustrates a cross-sectional view of the cannulated shaft of FIG. 7, taken generally along the line 8-8 of FIG. 7.

FIG. 8B illustrates a cross-sectional view of the cannulated shaft of FIG. 8A, taken generally along the line 8B-8B of FIG. 8A.

FIG. 9A illustrates a cross-sectional view of another embodiment of a cannulated shaft.

FIG. 9B illustrates a cross-sectional view of the cannulated shaft of FIG. 9A, taken generally along the line 9B-9B of FIG. 9A.

FIG. 10 illustrates a plan view of an embodiment of a cannulated shaft.

FIG. 11 illustrates a side view of an embodiment of a deployment shaft.

FIG. 12 illustrates a partial cross-sectional view of the embodiment of a deployment shaft of FIG. 11 disposed through a cannulated shaft and engaged therewith at a distal end thereof.

FIG. 13 illustrates a radially expandable region of a cannulated shaft plastically deformed.

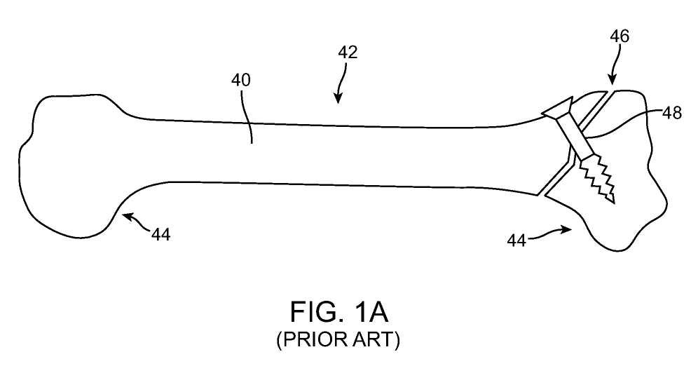

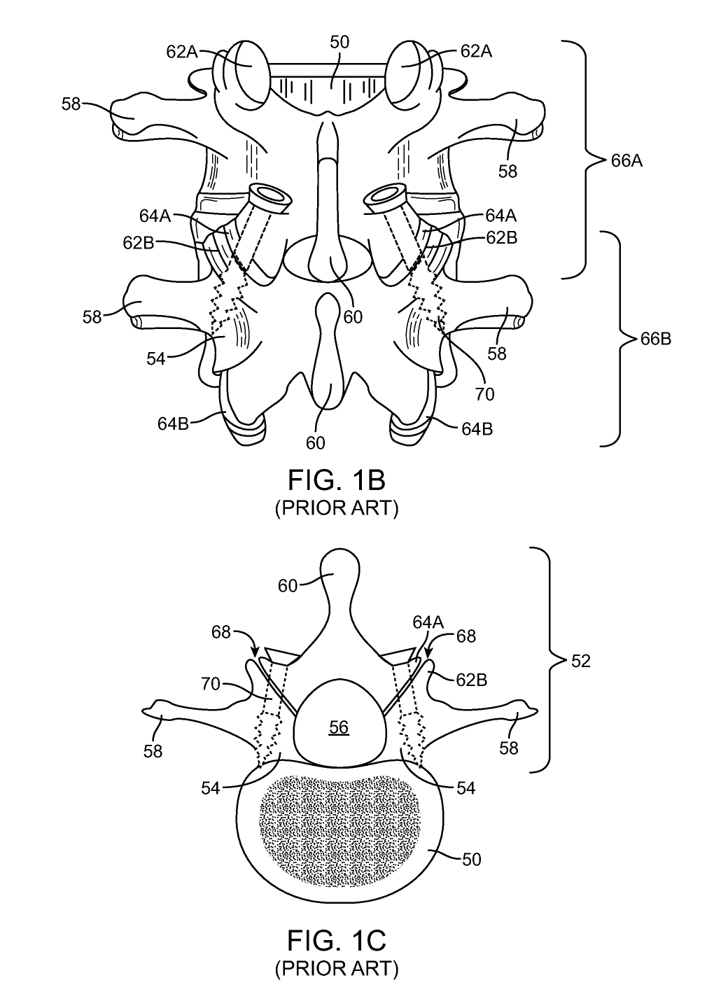

The foregoing and other features and advantages of the invention are apparent from the following detailed description of exemplary embodiments, read in conjunction with the accompanying drawings; wherein like structural or functional elements may be designated by like reference numerals.

### DETAILED DESCRIPTION

The words proximal and distal are applied to denote specific ends of components of the current invention described herein. A proximal end refers to the end of a component nearer to a medical professional when the component is implanted. A distal end refers to the end of a component further from the medical professional when the component is implanted.

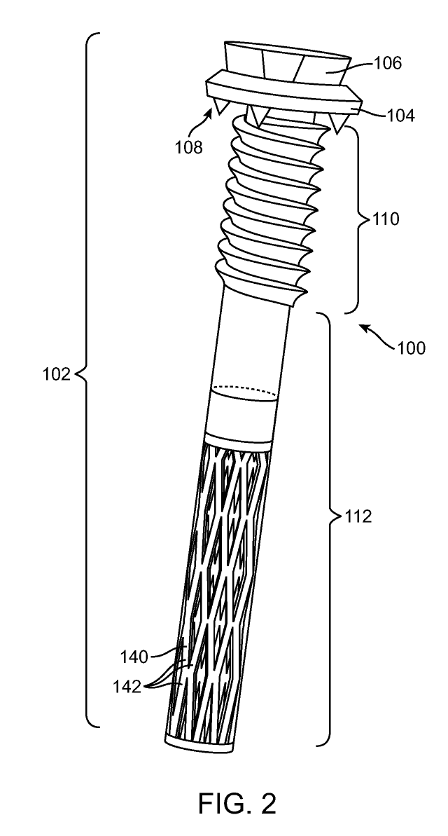

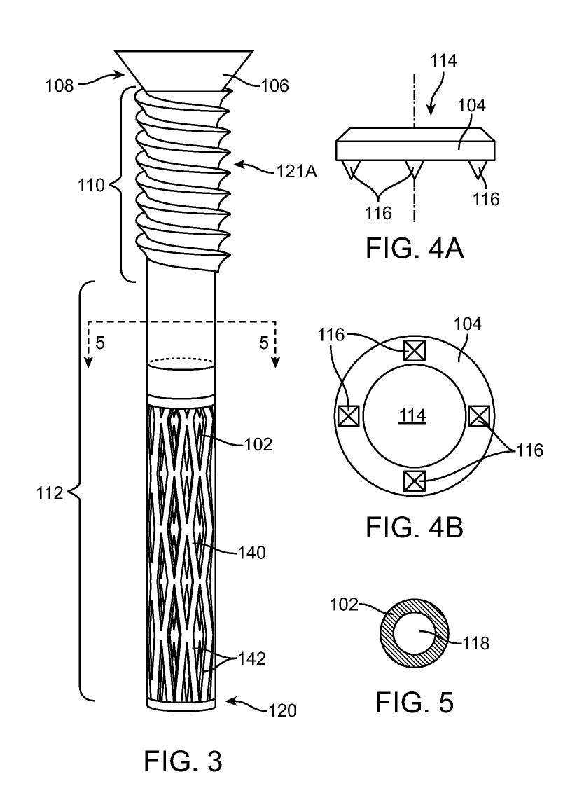

Referring to FIG. 2, an embodiment of a facet screw 100 having improved bone-grasping features includes a cannulated shaft 102 and a washer 104. Referring to FIG. 3, the cannulated shaft 102 includes a head 106 that is diametrically larger than the rest of the shaft 102 and that is disposed at a proximal end 108 of the shaft 102. The shaft 102 includes an externally threaded region 110 distal to the head 106 and a radially expandable region 112 distal to the externally threaded region 110. The cannulated shaft 102 may have a length between about 25 mm and 100 mm, preferably between about 25 mm and about 75 mm, and more preferably between about 25 mm and about 50 mm.

Referring to FIGS. 4A and 4B, the washer 104 includes a longitudinally disposed central opening 114 diametrically smaller than the diameter of the head 106 such that upon placement of the washer 104 over the shaft 102, the washer 104 is inhibited from sliding proximally off the shaft 102 over the head 106. In one embodiment, the washer 104 includes at least one protrusion 116 extending longitudinally therefrom. The at least one protrusion 116 may have, for example, a pyramidal shape, as illustrated in FIG. 4B, or any shape as may be desired, including by way of example and not limitation, a conical shape, a semispherical shape, a truncated conical shape, and the like. By orienting the washer 104 on the shaft 102 such that the at least one protrusion 116 is directed distally upon implantation of the facet screw 100, the at least one protrusion 116 may engage a surface of an inferior facet thereby providing an improved purchase with the inferior facet.

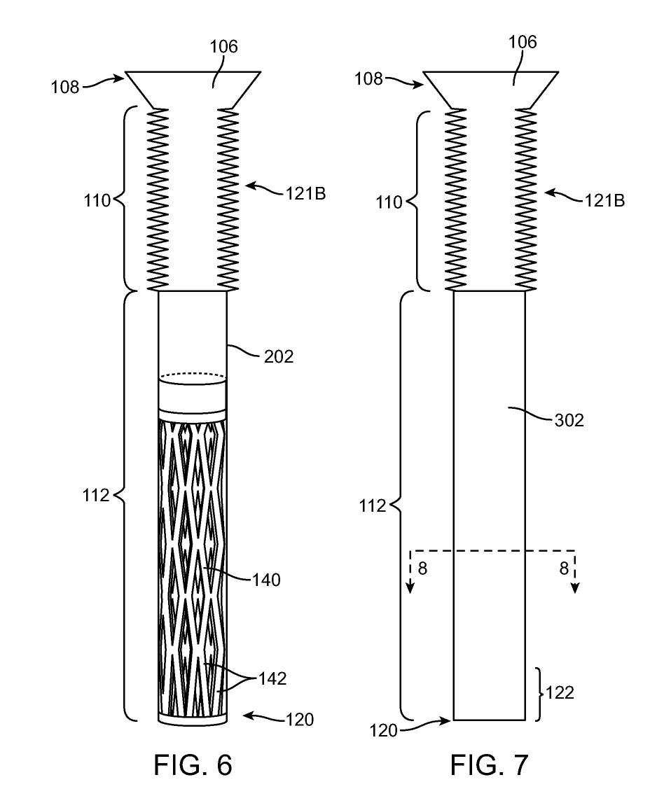

Referring to FIG. 5, the cannulated shaft 102 includes a longitudinal bore 118 extending therethrough from the head 106 to proximate a distal end 120 of the shaft 102, as will be described in more detail hereinbelow. In one embodiment, referring to FIG. 3, the externally threaded region 110 of the cannulated shaft 102 may comprise coarse cancellous threads 121A that may be preferable for use with cancellous bone, because the cancellous threads 121A may provide an improved grasp or grip when driven into sparse or thin bone. In other embodiments, as illustrated in FIGS. 6 and 7, the externally threaded region 110 of cannulated shafts 202, 302 may comprise fine cortical threads 121B that may be preferable for use with dense bone, because the cortical threads 121B may provide an improved grasp or grip when driven into dense bone. In other embodiments, the size and/or pitch of the threads within the externally threaded region 110 may be suitably tailored for specific needs particular to a given application as desired.

Referring to FIGS. 3, 6, and 7, in one embodiment, the externally threaded region 110, and the radially expandable region 112 of the cannulated shafts 102, 202, 302 may comprise a common material. In other embodiments, the externally threaded region 110 and the radially expandable region 112 of the cannulated shafts 102, 202, 302 may comprise different materials. Materials for the externally threaded region 110 and the radially expandable region 112 may be a suitable material as known in the art, including by way of example and not limitation stainless steel, nitinol, titanium, other shape memory metal materials, other metals, plastic, synthetic material, other suitable materials, or any combination thereof.

In a further embodiment, the radially expandable region 112 may be detachable from the externally threaded region 110. In this embodiment, the detachable regions may be attached by a suitable attachment mechanism, including by way of example and not limitation, a snap fit, threads, adhesives, or a bayonet socket. Such a cannulated shaft manufactured as separate externally threaded and radially expandable components may benefit from providing a medical professional the ability to tailor a suitable or desired type of threads in combination with a suitable or desired structure for the radially expandable region 112, as described in detail hereinbelow.

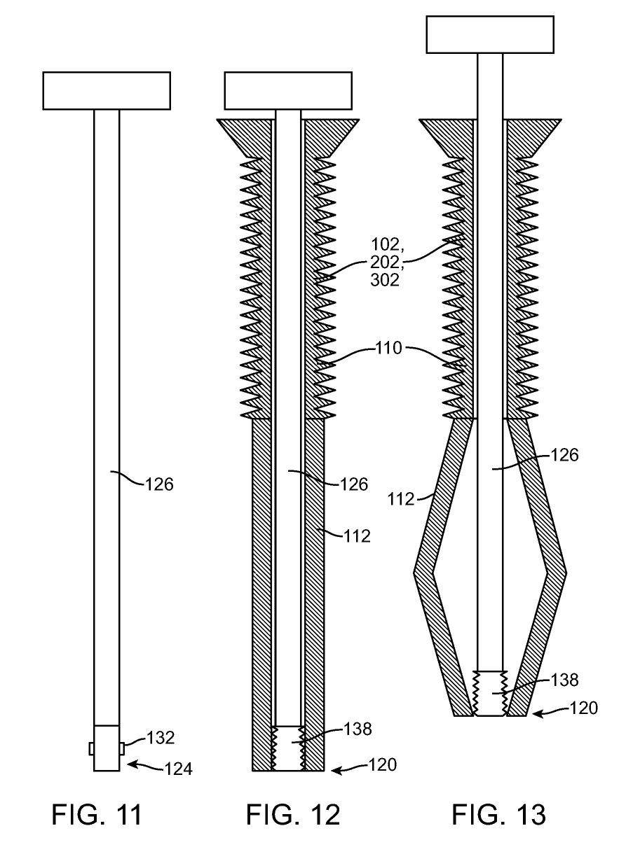

Referring to FIGS. 7-9B, the cannulated shaft 102, 202, 302 includes an attachment region 122 disposed proximate to the distal end 120 of the radially expandable region 112. Referring to FIGS. 11-13, the attachment region 122 is adapted to engage a distal end 124 of a deployment shaft 126 that is disposed through the bore 118 of the cannulated shaft 102, 202, 302. Engagement of the deployment shaft 126 and the attachment region 122 may comprise any suitable method or mechanism for attachment, including by way of example and not limitation, a bayonet socket, a cross key, or threads.

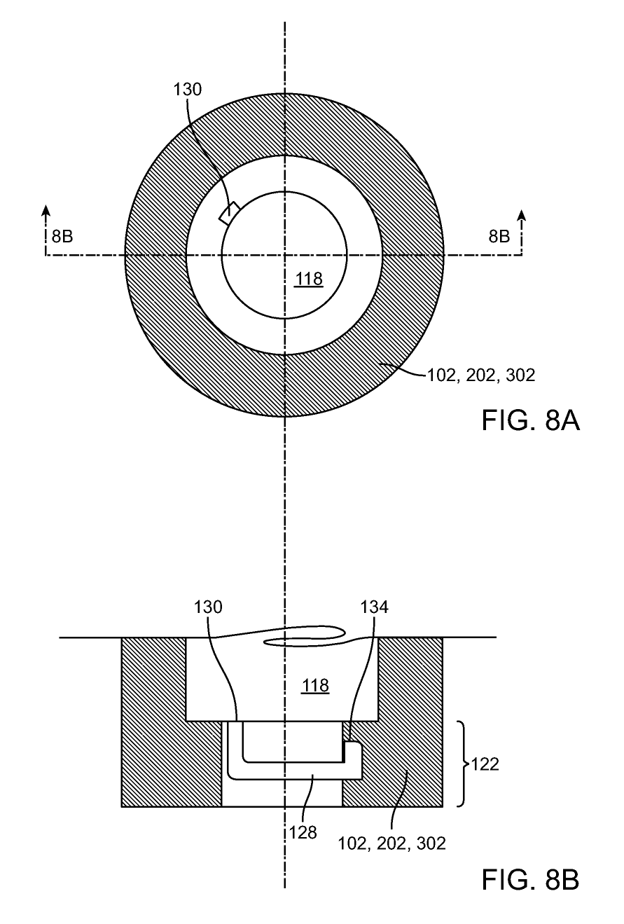

For example, referring to FIGS. 8A, 8B, and 11-13, in one embodiment, the attachment region 122 includes an internal groove 128 including an open end 130 directed proximally. A radial protrusion 132 disposed proximate the distal end 124 of the deployment shaft 126 is adapted to fit into the groove 128 and maneuvered such that the protrusion 132 is seated in a closed end 134 of the groove 128. With the protrusion 132 so seated, proximal force may be directed through the deployment shaft 126 to the distal end 120 of the radially expandable region 112.

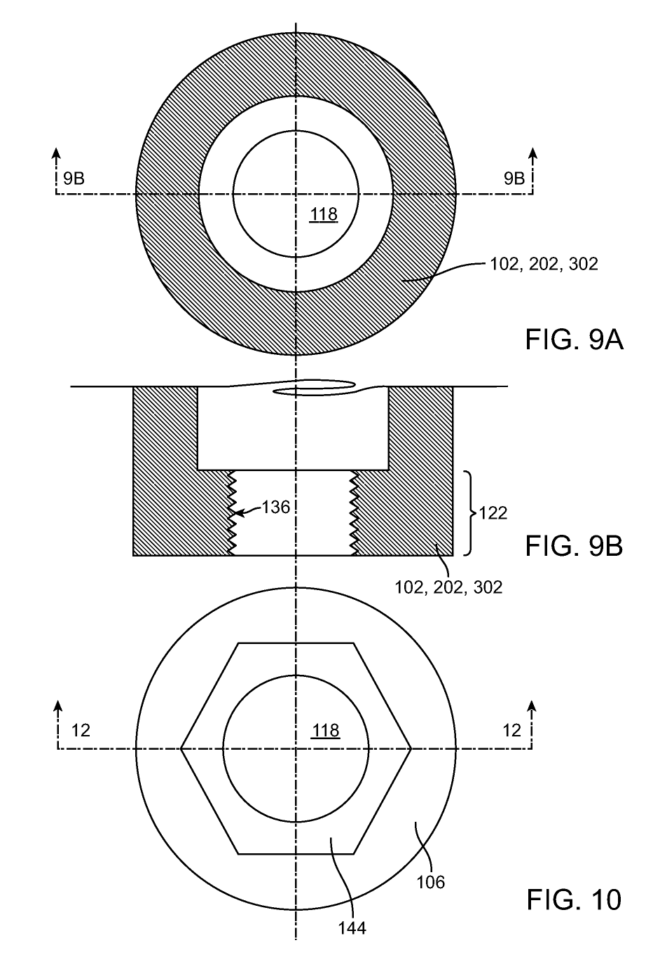

Referring to FIGS. 9A, 9B, and 11-13, in another embodiment, for example, the attachment region 122 includes an internally threaded region 136 disposed proximate the distal end 120 of the radially expandable region 112. External threads 138 disposed proximate the distal end 124 of the deployment shaft 126 are adapted to engage the internally threaded region 136 such that force may be directed through the deployment shaft 126 to the distal end 120 of the radially expandable region 112. Sufficient force proximally applied by the deployment shaft 126 to the attachment region 122 at the distal end 120 of the radially expandable region 112 causes longitudinal compression and consequent plastic radial expansion of the radially expandable region 112, as illustrated schematically in FIG. 13. Plastic radial expansion of the radially expandable region 112 provides that the radially expandable region 112 remains radially expanded upon detachment and removal of the deployment shaft 126 therefrom.

The radially expandable region 112 may comprise a structure as desired or suitable for the particular application as noted hereinabove with regard to the benefits of a two-piece cannulated shaft. In some embodiments, the radially expandable region 112 may, for example, comprise a cage structure 140, as schematically illustrated in FIGS. 2, 3, and 6. The cage structure 140 may comprise a plurality of thin struts 142 joined together or integrally manufactured to form a housing having spaces between the struts. The cage structure 140 may benefit from needing a reduced quantity of force to cause longitudinal compression and subsequent plastic radial expansion thereof. The cage structure 140 may further benefit from including spaces between the struts 142 to facilitate passage of bone cement or other fluid that may be forced through the bore 118 after expansion of the cage structure 140. Other embodiments of the radially expandable region 120 may include other structures, for example, a smooth spaceless rigid housing, a corrugated rigid housing, or other structure.

In use, in one embodiment, a hole is drilled and tapped through a first bone, for example, an inferior facet of a vertebra on a left side of the vertebra, and drilled into an adjacent second bone, for example, a superior facet and a pedicle on a left side of an adjacent vertebra. The cannulated shaft 102 including the washer 104 is driven through the first facet and into the second facet via the head 106 with a drive device, for example, a hexagonal head drive device (not shown). The head 106 includes a socket 144, for example the hexagonal socket 144 illustrated in FIG. 10 to accommodate the drive device. Following placement of the cannulated shaft 102, the deployment shaft 126 is introduced through the bore 118 and engaged with the attachment region 122, as described hereinabove with regard to FIGS. 8, 9, and 11-13. Proximal force is applied to the deployment shaft 126 to compress the radially expandable region 120 and thereby plastically radially expand the radially expandable region 120 within or below the second facet. Following expansion of the radially expandable region 120, the deployment shaft 126 is removed from the cannulated shaft 102, 202, 302. Typically, if the first and second bones are vertebra, the implantation procedure is repeated on a right side of the vertebrae (on a contra lateral side of the spine). Optionally, bone cement may be delivered through the cannulated shaft 102, 202, 302 to the radially expanded region. The cement can be radiopaque and can rapidly harden after application. The cement can also have a sufficiently low viscosity to allow injection of the cement through an appropriate cannula to the damage site. One example of bone cement that is suitable for use with certain embodiments disclosed herein is the OsseoFix+ Radiopaque Bone Cement available from Alphatec Spine, Inc.

A facet screw having features that facilitate improved relative fixation of any type of human and non-human bones is presented. The facet screw benefits from having a washer with protrusions extending from a distal side thereof and from a radially expandable region at a distal end of a cannulated shaft. An externally threaded region on the cannulated shaft benefits from having coarse or fine threads as desired by a medical professional for a particular application. The cannulated shaft further benefits from including a two piece embodiment that further facilitates selection of component parts to tailor the facet screw for a particular application.

It will be appreciated by those skilled in the art that changes could be made to the embodiments described hereinabove without departing from the broad concepts disclosed therein. It is understood, therefore, that this disclosure is not limited to the particular embodiments disclosed, but it is intended to cover modifications that may include a combination of features illustrated in one or more embodiments with features illustrated in any other embodiments. Various modifications, equivalent processes, as well as numerous structures to which the present disclosure may be applicable will be readily apparent to those of skill in the art to which the present disclosure is directed upon review of the present specification. Accordingly, this description is to be construed as illustrative only and is presented for the purpose of enabling those skilled in the art to make and use the facet screw described herein and to teach the best mode of carrying out the same.

The invention claimed is:

1. A facet screw for joining a first facet of a first vertebra with a second facet of a second vertebra, comprising:
a proximal end with a threaded region for attachment to the first facet; and
a distal end with a non-threaded region having a first configuration that freely inserts into a bore of the second facet and a second configuration that partially interferes with the bore to retain the distal end within the second facet.

2. The facet screw of claim 1, further comprising a cannula extending from the proximal end to the distal end.

3. The facet screw of claim 2, further comprising a deployment member that extends through the cannula and applies a compressive force to the distal end to transition the non-threaded region from the first configuration to the second configuration.

4. The facet screw of claim 3, wherein the deployment member removably attaches to the distal end.

5. The facet screw of claim 4, wherein the deployment member includes an external thread that engages with an internal thread of the cannula.

6. The facet screw of claim 4, wherein the deployment member includes a radial protrusion that engages with a groove of the cannula.

7. The facet screw of claim 1, wherein the threaded region and the non-threaded region comprise different materials.

8. The facet screw of claim 1, wherein the threaded region and the non-threaded region comprise a common material.

9. The facet screw of claim 1, wherein the non-threaded region is detachable from the threaded region.

10. The facet screw of claim 1, further comprising a head on the proximal end for driving the facet screw into the first and second facets.

11. The facet screw of claim 1, further comprising a washer coupled to the proximal end and having a protrusion extending distally for engagement with the first facet.

12. The facet screw of claim 1, wherein the non-threaded region comprises an expandable region and an attachment region distal to the expandable region.

13. The facet screw of claim 12, wherein the expandable region includes a plurality of struts forming an expandable cage.

14. The facet screw of claim 13, wherein the expandable cage directs flow of bone material injected into the second bore in the second configuration.

15. A facet screw having a proximal end and a distal end, comprising:
a cannulated shaft having:
a head disposed on the proximal end and having a diameter larger than a diameter of the cannulated shaft;
an externally threaded region distal to the head and configured for attachment to an inferior facet of a superior vertebra;
an expandable region distal to the externally threaded region and configured for insertion into bore of an adjacent superior facet of an inferior vertebra; and
an attachment region distal to the expandable region;
a washer coupled to the cannulated shaft distal to the head and having a protrusion extending from a distal end for engagement with the inferior facet; and
a deployment member having:
a proximal portion extending proximally from the head; and
a distal portion extending through the cannulated shaft and attaching to the attachment region, wherein the deployment member applies a proximally directed force to compress the cannulated shaft and expand a portion of the expandable region.

16. A method of joining a first facet of a first vertebra with a second facet of a second vertebra, comprising the steps of:
aligning the first facet with the second facet;
forming a first bore in the first facet;
forming a second bore in the second facet axially aligned with the first bore;
forming a thread pattern on the first bore;
inserting a facet screw having a threaded region that engages the thread pattern of the first bore and a non-threaded region that freely inserts into the second bore; and
expanding the non-threaded region to partially interfere with the second bore and retain the distal end within the second facet.

17. The method of claim 16, further comprising the step of inserting a deployment member through a cannula in the facet screw and applying a compressive force to expand the non-threaded region.

18. The method of claim 16, wherein the step of expanding the non-threaded region includes increasing a diameter of the non-threaded region from a first diameter that is less than a diameter of the second bore to a second diameter that is greater than or equal to the diameter of the second bore.

19. The method of claim 16, further comprising the step of injecting bone material through a cannula in the facet screw into the second bore.

20. The method of claim 16, further comprising the step of injecting bone material between the first facet and the second facet.
